(19) United States
(12) Patent Application Publication
(10) Pub. No.: US 2008/0275467 A1
(43) Pub. Date: Nov. 6, 2008

(54) Intraoperative guidance for endovascular interventions via three-dimensional path planning, x-ray fluoroscopy, and image overlay

(75) Inventors: Rui Liao, Plainsboro, NJ (US); Ashraf Mohamed, Houston, TX (US); Yiyong Sun, Lawrenceville, NJ (US); Chenyang Xu, Allentown, NJ (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 12/110,415

(22) Filed: Apr. 28, 2008

Related U.S. Application Data
(60) Provisional application No. 60/915,520, filed on May 2, 2007.

(51) Int. Cl. A61B 17/00

(52) U.S. Cl. 606/130; 703/11

(57) ABSTRACT

A method for planning a path for an endovascular intervention includes acquiring and displaying 3D angiographic images; selecting a target on the displayed images; extracting a skeleton of a vascular tree from the displayed images; extracting a symbolic vessel path to the target based on the skeleton of the vascular tree; and overlaying and displaying the symbolic vessel path on 2D fluoroscopic images for guiding the endovascular intervention.

### CROSS-REFERENCE TO RELATED APPLICATION

This application claims the benefit of provisional application Ser. No. 60/915,520, filed May 2, 2007 in the United States Patent and Trademark Office, the entire contents of which are herein incorporated by reference.

### BACKGROUND OF THE INVENTION

1. Technical Field

The present disclosure relates to the use of X-ray C-arm systems in interventional radiology and cardiology, more specifically, to the guidance of endovascular procedures such as the embolization of a blood vessel feeding a tumor, or the placement of an endovascular device such as a stent.

2. Discussion of the Related Art

X-ray C-arms are routinely used in medicine to acquire images for diagnostic assessment of a patient's vascular structures, and for guidance during interventional therapeutic procedures.

In today's angiographic X-ray C-arm systems, guidance is provided through the overlay of two-dimensional (2D) images acquired during the intervention, or projections of three-dimensional (3D) images acquired before or during the intervention.

Fluoroscopy images are low dose X-ray projection images that are used to guide and monitor the progress of an interventional procedure, e.g. to navigate a guidewire, a catheter, or an endovascular device, such as a stent.

Angiograms are 2D X-ray projection images of vascular structures filled with a contrast agent, which is typically injected intra-arterially through a catheter.

Digital subtraction angiography (DSA) subtracts two X-ray images, one with and one without contrast injection. The background anatomy cancels out, and the blood vessels into which contrast flows are highlighted.

3D angiograms can be obtained by rotating the X-ray C-arm around the patient's body for acquiring a set of angiograms as 2D projection images during the rotational run. A 3D volume image is reconstructed from this set of projections.

C-arm CT is a 3D image acquired by rotating a C-arm around the patient with or without contrast in the blood vessels. C-arm CT provides CT-like image quality inside the interventional suite.

During an endovascular intervention, the physician navigates a catheter or interventional device through the lumen of blood vessels towards an intended target inside the patient's vasculature.

The path from the entry site, typically a puncture wound leading to a major blood vessel, to the target is traditionally planned on 2D angiographic images. These images provide a “roadmap” for the intervention, and are typically superimposed on live fluoroscopic images for visual reference.

Branching and over-crossing blood vessels on a 2D roadmap image may make it difficult to discern individual blood vessels and to decide a path that a guidewire or catheter will follow.

Additionally, a single projection angle may make it difficult to know whether the catheter is truly close to the target or not. To overcome this limitation of 2D images, physicians may acquire images at multiple discrete orientations or inspect 3D images in comparison with the 2D fluoroscopic images.

Recent advancements in angiographic technology allow the overlay of 2D images created from 3D diagnostic or interventional images on 2D fluoroscopy images during an intervention.

3D angiography images, subtracted or non-subtracted, used for guidance during an interventional procedure may contain structures that could be distracting and not useful in guiding the physician to the target site in the vasculature via a selected path.

These potential distractions may be blood vessels other than the selected path, or structures, such as bone and soft tissue if the used angiographic images are non-subtracted. These structures may overlap with the planned path making the navigation cumbersome.

Overlapping structures in superimposed 3D images may further require the rotation of the C-arm to acquire fluoroscopic images at different orientations in order to resolve ambiguities or avoid an overlapping structure, thereby resulting in a cumbersome navigation

### SUMMARY OF THE INVENTION

A method for planning a path for an endovascular intervention, according to an exemplary embodiment of the present invention, includes acquiring and displaying 3D angiographic images; selecting a target on the displayed images; extracting a skeleton of a vascular tree from the displayed images; extracting a symbolic vessel path to the target based on the skeleton of the vascular tree; and overlaying and displaying the symbolic vessel path on 2D fluoroscopic images for guiding the endovascular intervention.

The 3D angiographic images may be acquired, prior to the intervention, by a computer tomography angiography (CTA) or a magnetic resonance angiography (MRA) imaging technique.

The 3D angiographic images may be acquired, during the intervention, by a C-arm CT or a 3D digital subtraction angiography (DSA) imaging technique.

The symbolic vessel path may be represented via vascular centerlines and branching points.

The method may further include selecting a source on the displayed images, and the symbolic vessel path may be extracted from the source to the target. When the source is not selected, the source may be defined as a main vessel.

The target may be a vessel of the vascular tree feeding a tumor.

The endovascular intervention may include guiding a catheter or a guidewire via the symbolic vessel path.

Audiovisual signals may be used for guiding the endovascular intervention or magnetic signals may be used for guiding the endovascular intervention.

The vascular intervention may be an embolization procedure.

A method for planning a path for an endovascular intervention, according to an exemplary embodiment of the present invention, includes acquiring and displaying 3D angiographic images; selecting a source and a target on the displayed images; extracting a symbolic vessel path, from the source to the target, from the displayed images; and overlaying and displaying the symbolic vessel path on 2D fluoroscopic images for guiding the endovascular intervention. The symbolic vessel path is represented via vascular centerlines and branching points along the path.

The 3D angiographic images may be acquired by a computer tomography angiography (CTA), a magnetic resonance angiography (MRA), a C-arm CT, and a 3D digital subtraction angiography (DSA) imaging technique.

The endovascular intervention may include guiding a catheter or a guidewire via the symbolic vessel path.

A computer system, according to an exemplary embodiment of the present invention, includes a processor; and a program storage device readable by the computer system, embodying a program of instructions executable by the processor to perform method steps for planning a path for an endovascular intervention, the method including acquiring and displaying 3D angiographic images; selecting a target on the displayed images; extracting a skeleton of a vascular tree from the displayed images; extracting a symbolic vessel path to the target based on the skeleton of the vascular tree; and overlaying and displaying the symbolic vessel path on 2D fluoroscopic images for guiding the endovascular intervention.

### BRIEF DESCRIPTION OF THE DRAWINGS

A more complete appreciation of the present disclosure and many of the attendant aspects thereof will be readily obtained, as the same becomes better understood by reference to the following detailed description, when considered in connection with the accompanying drawings, wherein:

FIG. 1 is a flowchart illustrating a method for displaying a planned vessel path for guidance during an intervention, according to an exemplary embodiment of the present invention;

FIG. 2 is a view of an extracted vessel path superimposed on an angiographic image, according to an exemplary embodiment of the present invention;

FIG. 3 is a diagram of a vessel tree showing a symbolic vessel path, according to an exemplary embodiment of the present invention; and

FIG. 4 shows an example of a computer system capable of implementing the method and apparatus according to exemplary embodiments of the present disclosure.

### DETAILED DESCRIPTION OF EXEMPLARY EMBODIMENTS

In describing exemplary embodiments of the present disclosure illustrated in the drawings, specific terminology is employed for the sake of clarity. However, the present disclosure is not intended to be limited to the specific terminology so selected, and it is to be understood that each specific element includes all technical equivalents which operate in a similar manner.

Exemplary embodiments of the present invention seek to provide:

A method and apparatus for identifying and isolating vascular structures of interest, and for facilitating the navigation of an endovascular catheter or device guidewire during endovascular interventional procedures.

A method for identifying and isolating vascular structures of interest in three-dimensional images from overlapping and occluding structures that are not directly useful for catheter or device guidewire navigation during a vascular interventional procedure. The extracted vascular structures form a vascular tree and may be represented via vascular centerlines, branching points, sizes of vessels along the centerline, and image intensity information for all parts of the vessels visible in the images.

An approach for extracting a path that represents the desired or planned path for catheter or device guidewire navigation inside the extracted vascular tree. The path is defined from the main vessel to one or more target vessels for the vascular intervention.

An approach for symbolically representing the extracted path inside a vascular tree and tree branching points for visualization of the path on two-dimensional or three-dimensional medical images.

An approach for visually assisting the navigation of an endovascular catheter or device guidewire inside the blood vessels of a patient during a surgical procedure via the image overlay of the extracted symbolic path on two dimensional medical images, including but not limited to X-ray fluoroscopic and angiographic images.

An approach to overlay the extracted symbolic path in addition to part or all of the original three-dimensional imaging data on two-dimensional medical images, including but not limited to X-ray fluoroscopic and angiographic images. The overlaid part of the medical image may include, but is not limited to, the aforementioned vascular tree, or any part thereof.

An approach for live (on-line) automatic update of the three-dimensional images or sub-images showing the symbolic representation of the vascular tree and the vascular path on two-dimensional medical images, including but not limited to X-ray fluoroscopy and angiography images.

In conjunction with the use of a system for tracking an endovascular catheter or device guidewire, including but not limited to an optical, magnetic, or image-based tracking system, a method for machine-based audible or visual confirmation in case the catheter or guidewire stays on the planned path, or audible/visible warning in case the catheter or guidewire deviates from it.

In conjunction with the use of a steerable catheter or guidewire navigation device, including but not limited to magnetic, mechanical or electrical navigation, an approach for using the extracted path to derive electrical, magnetic, or mechanical signals that will steer the catheter or guidewire in order to ascertain that it stays on the planned path at vessel bifurcation points, for example.

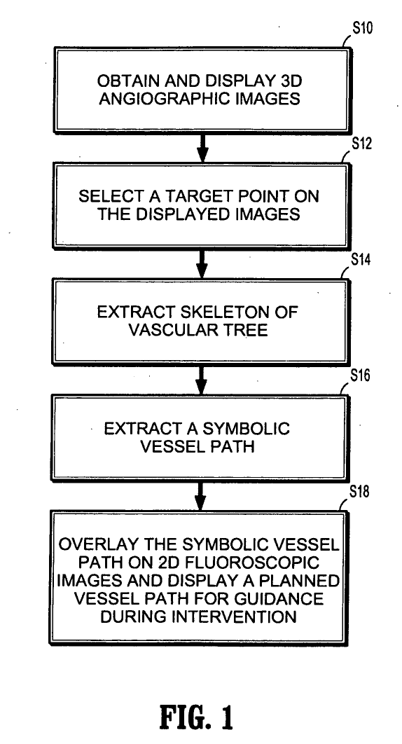

FIG. 1 is a flowchart illustrating a method for displaying a planned vessel path for guidance during an intervention, according to an exemplary embodiment of the present invention.

3D angiographic images are obtained and displayed (Step S10). These 3D images may be acquired prior to the intervention, by using systems such as Computed Tomography Angiography (CTA) and Magnetic Resonance Angiography (MRA), or while the patient is in the interventional suite, by using systems such as C-arm CT or 3D DSA.

A user selects a target point inside blood vessels on the displayed angiographic images (Step S12). The user may also select a starting or source point for the navigation inside the vasculature. Coordinates of the two selected points may be stored.

A skeleton of a vascular tree is automatically extracted (Step S14). A vessel skeleton may be represented as a set of nodes connected by line segments. The distance between two nodes is usually one voxel in the input image data.

A vessel branch is composed of a series of nodes and line segments in between. A node is a branching node if it has more than two line segments associated with it. A branching node is therefore associated with the branching of a vessel.

A node is termed a terminal node if it has only one line segment associated with it. A vessel diameter and vessel orientation 3D vector may be associated with each node in the skeleton. The vessel skeleton and associated vessel radius and orientation may be found by one of a variety of vessel skeletonization and vessel representation algorithms.

A symbolic vessel path is extracted by finding a path from the target point back to the source point, or to the main vessel if a source point was not selected by the user (Step S16). The symbolic vessel path is extracted from the extracted vascular tree or, alternatively, directly from the displayed images.

From all the nodes in the vessel skeleton, two nodes are termed the target node and the source node. These two nodes are found by computing distances from the user selected target and starting points to all nodes in the vessel skeleton.

The two nodes with minimal distances to the target and source points are identified as the target and source nodes respectively. If the user has not selected a source point, the source node is identified as the terminal node in the vessel tree with the largest associated vessel diameter.

If the vessel tree has no cycles or closed paths, there exists a unique path from the target point to the source point. Therefore, a path through the vessels may be represented via the portion of the skeleton that connects the target node back to the source node.

Using the structure of the vessel skeleton tree, an algorithm can automatically traverse the unique path from the target point back to the source point.

Through knowledge of the projection geometry of the C-arm system and the geometric transformation from the 3D angiographic dataset to the C-arm position, a symbolic 3D representation of the selected path is superimposed on 2D fluoroscopic images, displayed, and used for guidance during the intervention (Step S18). The user may modify the view of the path and adjust the degree of blending of the path with the fluoroscopic image.

An exemplary embodiment of the present invention makes use of knowledge of the projective geometry of a fluoroscopic C-arm in order to superimpose the symbolically represented vessel path on 2D images.

For example, let the Cartesian coordinates of a point in 3D space be given by the triplet (x,y,z) and let the image coordinates of the same point be (u,v) in pixels. The relationship between the two sets of coordinates is given by equation (1):

$$[ \begin{matrix}α u \\ α v \\ α\end{matrix} ] = P [ \begin{matrix}x \\ y \\ z \\ 1\end{matrix} ] \qquad ( 1 )$$

Where α is a scalar and P is a 3×4 matrix with 10 degrees of freedom. The parameters of P may be obtained through knowledge of the geometric design parameters of the C-arm, system calibration procedure, and the location of the C-arm.

Other alternative approaches for achieving 2D to 3D registration will be obvious to those skilled in the art.

The set of line segments connecting the nodes of the planned endovascular path is projected onto the 2D fluoroscopic image.

In an exemplary embodiment of the present invention, nodes along the planned vessel path are not displayed in image overlay with two exceptions. First, the source and target nodes of the selected path are displayed with a color that is different from that used to display line segments of the path, for example. Second, branching nodes along the selected path are also superimposed on the 2D images. Displayed nodes may be represented via small spheres with a fixed radius. Branching nodes help provide the user with knowledge of branching points along the path, where there is a possibility that the manipulated catheter or device may veer off path.

In an exemplary embodiment of the present invention, the user may switch to viewing the selected path based on a 3D image. Given the skeleton of the selected path, and the diameter of the vessels associated with every node on the path, it is possible to segment the vessels that constitute the path out of the 3D image.

By way of example, voxels from the 3D volume that are within a certain distance around the skeleton nodes of the selected path are displayed in a volume rendered view and superimposed on the 2D fluoroscopic image. This distance may be derived from the vessel diameter associated with the path nodes. This approach provides a fast and efficient segmentation of the relevant structures from the 3D volume and their use for guidance during the intervention.

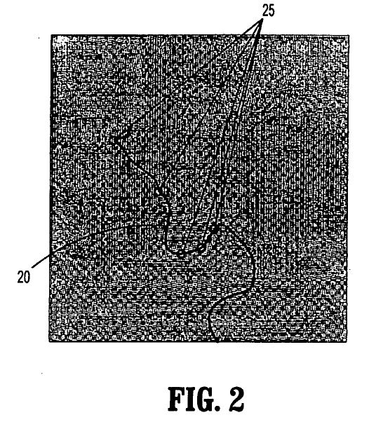

FIG. 2 is a view of an extracted vessel path 20 superimposed on an angiographic image from a liver chemo embolization procedure, according to an exemplary embodiment of the present invention. Vessel branching points 25 are represented by spheres.

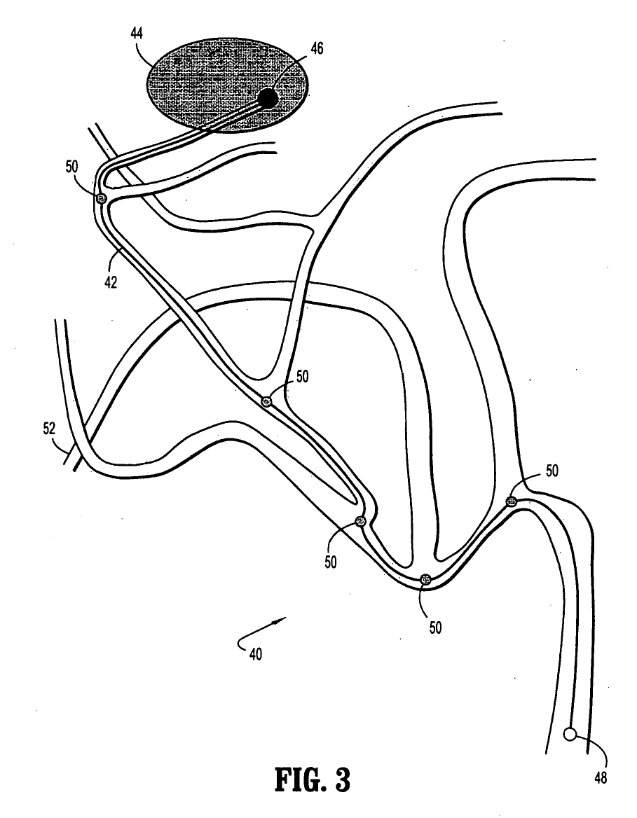

FIG. 3 is a diagram of a vessel tree 40 showing a symbolic vessel path 42, according to an exemplary embodiment of the present invention, and shows a vessel tree as would be available from a 3D angiographic medical image showing blood vessels, e.g., MRA, 3D X-ray Angiography, or CT angiography.

The oval 44 is a structure of interest, such as a tumor that is fed by some branches of the vessel tree 40. Using the vessel tree 40 as a roadmap by overlaying it on 2D fluoroscopy or X-ray angiography may be confusing or unclear to the doctor, since the vessels have many branchings and may overcross each other when the images are viewed from a particular angle.

According to an exemplary embodiment of the present invention, the user marks the target blood vessel 46 for the catheter or guidewire on the 3D images. In this example, the target blood vessel 46 is the blood vessel that feeds the tumor 44. The user may also mark the main blood vessel 48, or it may be extracted automatically.

The symbolic vessel path 42 is extracted between the two dots 46 and 48, and shown as a line composed of small line segments and vessel branching points 50. Having this symbolic path overlaid on the 2D interventional images, such as X-ray angiography or fluoroscopy, helps the user navigate the catheter or guidewire and helps clarify where the branching points 50 are, and where the planned path 42 is, specially when there are overcrossing vessels, such as vessel 52.

Further, tracking of the catheter or the device used during the intervention may be achieved with a variety of technologies. By way of example, it is possible to determine a 3D location of a catheter tip via images from biplane angiographic C-arm systems. Another exemplary approach for tracking a catheter is through magnetic tracking methods that use a miniature electromagnetic sensor embedded in the catheter tip.

Given the 3D location of the tracked catheter or interventional device, an exemplary embodiment of the present invention is able to determine the distance between this location and the closest node in the selected path. If this distance is greater than a preset threshold (e.g., 5 mm or the maximum vessel diameter), an audible warning (e.g., an intermittent beeping sound) and/or a visual warning (e.g., flashing the catheter or device location via a small red colored sphere on the fluoroscopic image monitor), may be provided.

With such a warning, the user may be able to return the tracked catheter or device back to the last branching point in order to follow the correct branch along the planned path.

The user may also be alerted with an audible or visual indication when the tracked catheter or device is within a present small distance from the target node. By way of example, the system can change the color of the superimposed target node to flashing green when the tracked device is a few millimeters away from it. Additionally, an audible indication or a voice message may alert the user of this proximity to the target point.

Such audible and visual indicators are useful in cases where the used fluoroscopic 2D image projection has significant foreshortening that may provide an incorrect illusion of proximity.

Furthermore, magnetic path navigation devices have been proposed to enable the steering of a catheter in order to follow a planned path. In these devices, magnetic signals are input manually by the user, and signals are derived for steering and navigation that will help guide the catheter to a planned target location based on the three dimensional representation of the vessel path described above. In an exemplary embodiment of the present invention, at every branching point, where there is a chance for the catheter or device to veer off path, the magnetic steering signals may be derived so that the navigated catheter will follow the selected path branch.

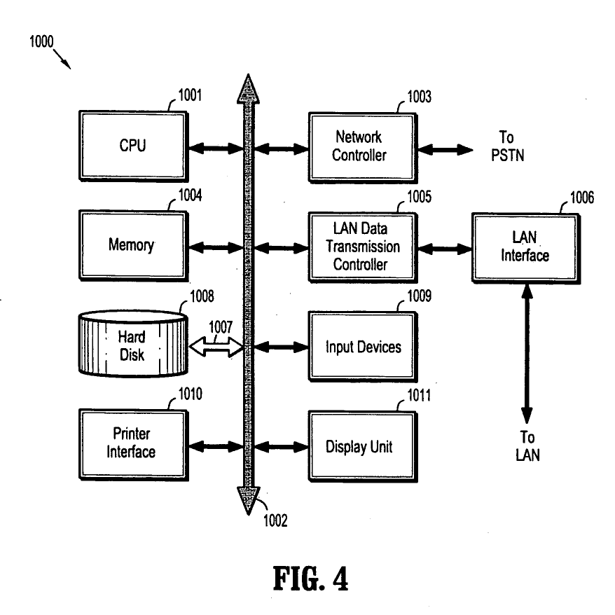

FIG. 4 shows an example of a computer system which may implement a method and system of the present disclosure. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007.

Having described exemplary embodiments of the present invention, it is to be understood that the invention is not limited to the disclosed embodiment, but, on the contrary, is intended to cover various modifications and equivalent arrangements included within the spirit and scope of the disclosure.

What is claimed is:

1. A method for planning a path for an endovascular intervention, comprising:
acquiring and displaying 3D angiographic images;
selecting a target on the displayed images;
extracting a skeleton of a vascular tree from the displayed images;
extracting a symbolic vessel path to the target based on the skeleton of the vascular tree; and
overlaying and displaying the symbolic vessel path on 2D fluoroscopic images for guiding the endovascular intervention.

2. The method of claim 1, wherein the 3D angiographic images are acquired, prior to the intervention, by one of a computer tomography angiography (CTA) and a magnetic resonance angiography (MRA) imaging technique.

3. The method of claim 2, wherein the 3D angiographic images are acquired, during the intervention, by one of a C-arm CT and a 3D digital subtraction angiography (DSA) imaging technique.

4. The method of claim 1, wherein the symbolic vessel path is represented via vascular centerlines and branching points.

5. The method of claim 4, further comprising selecting a source on the displayed images.

6. The method of claim 5, wherein the symbolic vessel path is extracted from the source to the target.

7. The method of claim 6, wherein when the source is not selected, the source is defined as a main vessel.

8. The method of claim 1, wherein the target is a vessel of the vascular tree feeding a tumor.

9. The method of claim 4, wherein the endovascular intervention includes guiding one of a catheter and a guidewire via the symbolic vessel path.

10. The method of claim 9, wherein audiovisual signals are used for guiding the endovascular intervention.

11. The method of claim 9, wherein magnetic signals are used for guiding the endovascular intervention.

12. The method of claim 9, wherein the vascular intervention is an embolization procedure.

13. A method for planning a path for an endovascular intervention, comprising:
acquiring and displaying 3D angiographic images;
selecting a source and a target on the displayed images;
extracting a symbolic vessel path, from the source to the target, from the displayed images; and
overlaying and displaying the symbolic vessel path on 2D fluoroscopic images for guiding the endovascular intervention, wherein the symbolic vessel path is represented via vascular centerlines and branching points along the path.

14. The method of claim 13, wherein the 3D angiographic images are acquired by one of a computer tomography angiography (CTA), a magnetic resonance angiography (MRA), a C-arm CT, and a 3D digital subtraction angiography (DSA) imaging technique.

15. The method of claim 14, wherein the endovascular intervention includes guiding one of a catheter and a guidewire via the symbolic vessel path.

16. A computer system comprising:
a processor; and
a program storage device readable by the computer system, embodying a program of instructions executable by the processor to perform method steps for planning a path for an endovascular intervention, the method comprising:
acquiring and displaying 3D angiographic images;
selecting a target on the displayed images;
extracting a skeleton of a vascular tree from the displayed images;
extracting a symbolic vessel path to the target based on the skeleton of the vascular tree; and
overlaying and displaying the symbolic vessel path on 2D fluoroscopic images for guiding the endovascular intervention.

17. The method of claim 16, wherein the symbolic vessel path is represented via vascular centerlines and branching points.

18. The method of claim 17, wherein the endovascular intervention includes guiding one of a catheter and a guidewire via the symbolic vessel path.

19. The method of claim 18, wherein the target is a vessel of the vascular tree feeding a tumor.

20. The method of claim 19, wherein the vascular intervention is an embolization procedure.
